(19) United States
(12) Patent Application Publication
(10) Pub. No.: US 2017/0108562 A1
(43) Pub. Date: Apr. 20, 2017

(54) RF SURFACE COIL UNIT AND MAGNETIC RESONANCE IMAGING SYSTEM COMPRISING SAME

(71) Applicant: Samsung Electronics Co., Ltd., Suwon-si (KR)

(72) Inventor: Kyoungnam KIM, Incheon (KR)

(73) Assignee: Samsung Electronics Co., Ltd., Suwon-si (KR)

(21) Appl. No.: 15/318,201

(22) PCT Filed: Jun. 10, 2015
(86) PCT No.: PCT/KR2015/005799

§ 371 Date: Dec. 12, 2016

(30) Foreign Application Priority Data
Jun. 12, 2014 (KR) 10-2014-0071487

(51) Int. Cl. G01R 33/341; G01R 33/36; A61B 5/055

(52) U.S. Cl. CPC G01R 33/341; A61B 5/0555; G01R 33/365

(57) ABSTRACT

A radio frequency (RF) surface coil unit and a magnetic resonance imaging (MRI) system including the same are provided. The RF surface coil unit includes multiple loop-shaped RF coil elements including at least one first RF coil element having a loop shape and at least one second RF coil element formed in the at least one first RF coil element. The at least one first RF coil element and the at least one second RF coil element are electrically connected to each other and formed as one channel.

### TECHNICAL FIELD

The present invention relates to a radio frequency (RF) surface coil unit for use in a magnetic resonance imaging (MRI) system and an MRI system including the RF surface coil unit.

### BACKGROUND ART

Diverse diagnostic apparatuses for diagnosing an abnormality in a physical body have been used to prevent and cure diseases. Among them, a magnetic resonance imaging (MRI) apparatus that uses a magnetic field generated by a magnetic force is widely used.

The MRI apparatus is a medical apparatus for acquiring a sectional image of a part of an object by expressing, via a contrast comparison, a strength of a magnetic resonance (MR) signal with respect to a radio frequency (RF) signal generated in a magnetic field having a specific strength. If an RF signal that resonates only a specific atomic nucleus (for example, a hydrogen atomic nucleus) is irradiated for a short period of time by using an RF coil onto the object that is placed in a strong magnetic field, and then the irradiation is discontinued, an MR signal is emitted from the specific atomic nucleus, and thus the MRI apparatus may receive the MR signal via the RF coil and acquire an MR image. An intensity of the MR signal may be determined according to a density of a predetermined atom (for example, hydrogen, sodium, or carbon isotopes) in the object or a blood flow.

The MRI apparatus may include the RF coil configured to transmit a high frequency signal and receive an MR signal. One RF coil may resonate a magnetization vector (in an RF transmission mode) and receive the MR signal (in an RF reception mode) at the same time. Also, two RF coils, namely, an RF transmission mode exclusive RF coil and an RF reception mode exclusive RF coil, may be used to separately operate in the RF transmission mode and the RF reception mode. The RF coil operating in both the transmission mode and the reception mode is referred to as a transmission and reception (Tx/Rx) coil, and the transmission exclusive coil and the reception exclusive coil are referred to as a transmission coil and a reception coil, respectively.

### DETAILED DESCRIPTION OF THE INVENTION

### Technical Problem

Embodiments disclosed herein relate to a magnetic resonance imaging (MRI) system including a radio frequency (RF) surface coil unit including at least one loop coil formed in a single loop coil in the MRI system. The technical problems to be solved by the present embodiments are not limited to the above-described one and other technical problems may further be solved.

### Technical Solution

According to an exemplary embodiment, a radio frequency (RF) surface coil unit for a magnetic resonance imaging (MRI) system, the RF surface coil unit comprising multiple RF coil elements comprising: at least one first RF coil element having a loop shape; and at least one second RF coil element formed in the at least one first RF coil element.

The at least one first RF coil element and the at least one second RF coil element may be electrically connected to each other, and the multiple RF coil elements comprising the at least one first RF coil element and the at least one second RF coil element may be formed as one channel.

The RF surface coil unit may comprise at least one channel comprising the multiple RF coil elements.

The at least one channel may be formed as a plurality of channels, and a decoupling device may be formed for decoupling between the coil elements of the at least one channel.

The decoupling device may comprise a capacitor.

The decoupling device may further comprise a decoupling circuit formed as an inductor or a transformer.

The decoupling device may comprise a decoupling circuit formed as an inductor or a transformer.

The at least one first RF coil element may have a circular shape, an oval shape, or a polygonal shape.

The at least one second RF coil element may have a circular shape, an oval shape, or a polygonal loop shape.

According to an exemplary embodiment, a magnetic resonance imaging (MRI) system comprising: a radio frequency (RF) surface coil unit comprising multiple RF coil elements comprising: at least one first RF coil element having a loop shape; and at least one second RF coil element formed in the at least one first RF coil element.

### Advantegeous Effects of the Invention

According to a magnetic resonance imaging (MRI) system of the disclosed embodiments, a radio frequency (RF) surface coil unit including multiple loop-type RF coil elements formed as one channel may have improved sensitivity and uniformity. The RF surface coil unit according to the disclosed embodiments may be applied to a transmission and reception (Tx/Rx) coil or a reception exclusive (Rx) MRI RF coil.

### DESCRIPTION OF THE DRAWINGS

FIG. 1 is a structural diagram of a magnetic resonance imaging (MRI) system according to an embodiment.

FIGS. 2A and 2B are views of a radio frequency (RF) surface coil unit of an MRI system, according to embodiments.

FIGS. 3A through 3C are views showing a direction of currents of an RF surface coil unit of an MRI system, according to an embodiment.

FIG. 4 is a view of an RF surface coil unit of an MRI system, wherein the RF surface coil unit is formed as four channels, according to an embodiment.

FIG. 5 is a view of an RF surface coil unit of an MRI system, wherein the RF surface coil unit is expanded as multiple channels, according to an embodiment.

### MODE OF THE INVENTION

Hereinafter, a radio frequency (RF) surface coil unit and a magnetic resonance imaging (MRI) system including the RF surface coil unit according to embodiments of the present invention will be described in detail by referring to the accompanying drawings. In the drawings, the thicknesses of layers and regions may be exaggerated for clarity. Like reference numerals refer to like elements throughout.

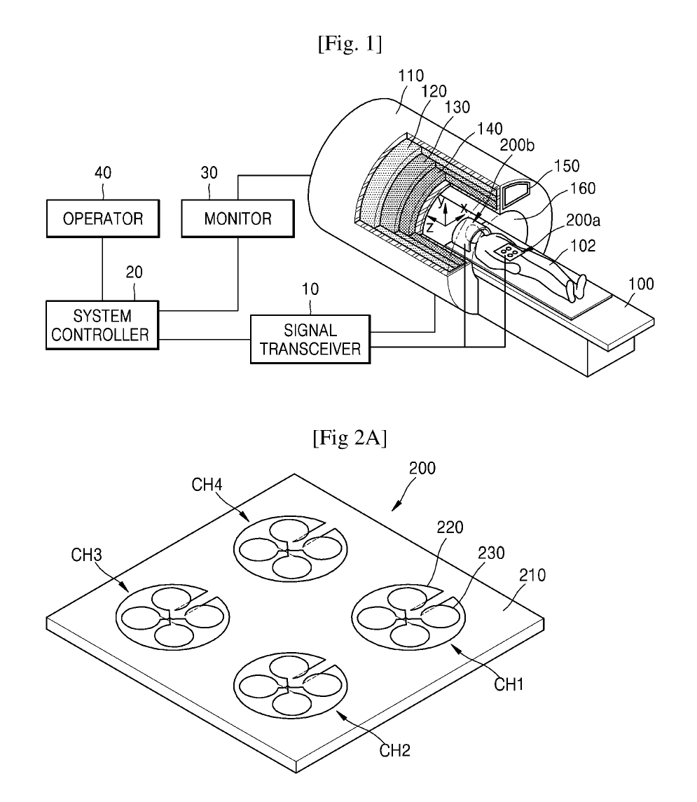

FIG. 1 is a structural diagram of an MRI system according to an embodiment.

Referring to FIG. 1, the MRI system according to the present embodiment may include a main magnet 120 mounted in a housing 110 of a cylindrical shape, a gradient coil unit 130, and an RF body coil unit 140.

The main magnet 120 may generate a magnetostatic field or a static magnetic field for aligning, in a constant direction, a direction of magnetic dipole moments of atomic nuclei of elements causing magnetic resonance, such as hydrogen, phosphorous, or sodium, from among elements distributed in an object 102. In the present specification, an “object” may include a person or an animal or a part of a person or an animal. For example, the object 102 may include the liver, the heart, the womb, the brain, the breast, the abdomen, or a blood vessel. The main magnet 120 may include a superconducting magnet or a permanent magnet. For example, the superconducting magnet may generate a high magnetic field that is equal to or higher than 0.5 T. As the magnetic field generated by the main magnet 120 is strong and uniform, a more precise and accurate magnetic resonance image with respect to the object 102 may be obtained. The main magnet 120 may have a cylindrical shape.

The gradient coil unit 130 may be formed at an inner side of the main magnet 120. The gradient coil unit 130 may include three gradient coils for generating gradient magnetic fields in X-, Y-, and Z-axis directions crossing each other at right angles. The gradient coil unit 130 may generate a spatially linear gradient magnetic field for photographing a magnetic resonance image. The gradient coil unit 130 may provide location information of each region of the object 102 by differently inducing resonance frequencies according to the regions of the object 102.

The RF body coil unit 140 may be mounted at an inner side of the gradient coil unit 130, and may be included in the cylindrical-shaped magnetic structure, together with the main magnet 120 and the gradient coil unit 130. RF surface coil units 200a and 200b may be formed adjacent to the object 102 on a table 100.

An RF coil device including the RF body coil unit 140 or the RF surface coil units 200a and 200b may generate a high frequency magnetic field having a Lamor frequency as the main frequency. The RF coil device may irradiate an RF signal onto the object 102 and receive a magnetic resonance signal emitted from the object 102. For example, in order to make an atomic nucleus transit from a low energy state to a high energy state, the RF coil device may generate and apply an electromagnetic wave signal having an RF corresponding to a type of the atomic nucleus, for example, an RF signal, to the object 102. When the electromagnetic wave signal generated by the RF coil device is applied to the atomic nucleus, the atomic nucleus may transit from the low energy state to the high energy state. Then, when electromagnetic waves generated by the RF coil device disappear, the atomic nucleus on which the electromagnetic waves were applied transits from the high energy state to the low energy state, thereby emitting electromagnetic waves having a Lamor frequency. In other words, when the applying of the electromagnetic wave signal to the atomic nucleus is stopped, an energy level of the atomic nucleus is changed from a high energy level to a low energy level, and thus the atomic nucleus may emit electromagnetic waves having a Lamor frequency. The RF coil device may receive electromagnetic wave signals from atomic nuclei in the object 102. The RF body coil unit 140 may be fixed at the inner side of the gradient coil unit 130 of the housing 110, and the RF surface coil units 200a and 200b may be detachable. The RF surface coil units 200a and 200b may be used to diagnose a specific region of the object 102, and may be used to diagnose a region of the object 102, wherein the region includes the head, the neck, the shoulder, the chest, the wrist, the leg, the ankle, etc. of the object 102.

The housing 110 including the main magnet 120, the gradient coil unit 130, and the RF body coil unit 140 may have a cylindrical shape. A bore 160, which is a space into which the table 100 on which the object 102 is located may enter, may be formed in the housing 110. The bore 160 may be formed in the Z-axis direction, and a diameter of the bore 160 may be determined according to sizes of the main magnet 120, the gradient coil unit 130, and the RF body coil unit 140.

A display 150 may be mounted at an outer side of the housing 110 of the MRI system. Also, a display may further be included at an inner side of the housing 110. Certain information may be transmitted to a user or the object 102 through the display(s) located at the inner side and/or the outer side of the housing 110.

Also, the MRI system may include a signal transceiver 10, a system controller 20, a monitor 30, and an operator 40.

The signal transceiver 10 may control a gradient magnetic field formed in the housing 110, that is, the bore 160, and may control transmission and reception of an RF signal and a magnetic resonance signal related to the RF body coil unit 140 and the RF surface coil units 200a and 200b. The system controller 20 may control signals generated in the housing 110. The monitor 30 may monitor or control the housing 110 or various devices mounted in the housing 110. The operator 40 may order pulse sequence information from the system controller 20, and control overall operations of the MRI system. The object 102, located on the table 100, may be inspected in a state in which the object 102 is moved and fixed in a direction in which the bore 102 is formed, that is, the Z-axis direction, or in a state in which the object 102 is being moved.

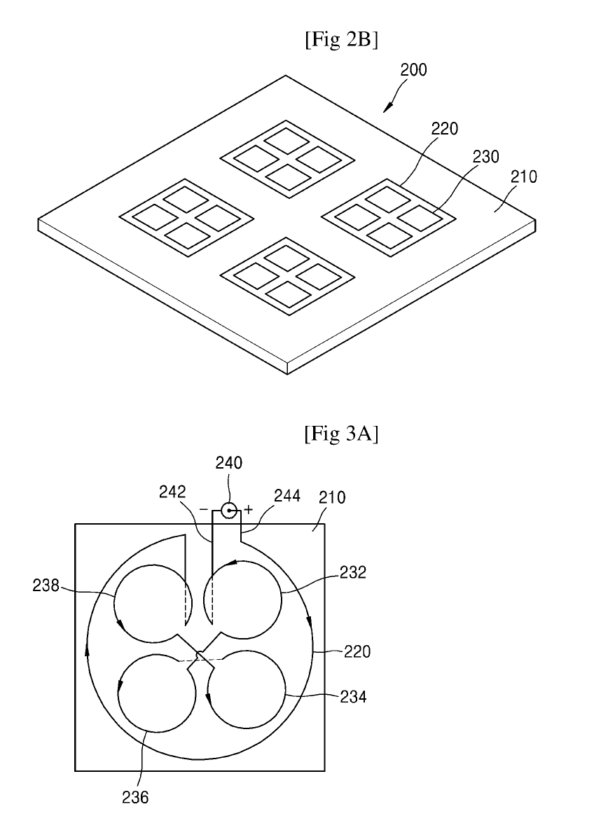

FIGS. 2A and 2B are views of an RF surface coil unit 200 of an MRI system, according to embodiments.

Referring to FIG. 2A, the RF surface coil unit 200 according to the present embodiment may include one or more RF coil elements 220 and 230 formed on a base 210. The RF coil elements 220 and 230 may include the first RF coil element 220 and at least one second RF coil element 230 formed in the first RF coil element 220. Each of the first RF coil element 220 and the second RF coil element 220 may be formed to have a loop shape, and the second RF coil element 230 may be formed in a loop of the first RF coil element 220. The number of second RF coil elements 230 formed in the first RF coil element 220 is not limited, and the number and size of second RF coil elements may vary according to the size and shape of the first RF coil element 220. Also, the location and size of the second RF coil elements 230 in the first RF coil element 220 may be set to adjust a distribution of a magnetic field according to a region of interest of the object 102, which is to be measured.

FIG. 2A illustrates that the first RF coil element 220 is formed to have a circular loop shape, and four second RF coil elements 230 are formed in the first RF coil element 220. However, it is an example, and the present invention is not limited thereto. For example, FIG. 2B illustrates that the first RF coil element 220 is formed to have a quadrangular loop shape, and a plurality of second RF coil elements 230 having quadrangular loop shapes are formed in the first RF coil element 220. The first RF coil element 220 and the second RF coil elements may have substantially the same shapes as illustrated in FIGS. 2A and 2B, or may have different shapes from each other. For example, the first RF coil element 220 may have a circular shape, while the second RF coil elements 230 may have a quadrangular shape. The RF coil elements 220 and 230 of the RF surface coil unit 200 according to embodiments are not limited to particular shapes and may have a circular shape, an oval shape, or a polygonal shape, such as a triangular shape, a quadrangular shape, etc.

As described above, the RF surface coil unit 200 according to the embodiments may have the structure in which one or more second RF coil elements 230 are further included in one loop-shaped first RF coil element 220. Here, the first RF coil element 220 and the second RF coil elements 230 may be electrically connected to each other and form a channel. That is, according to the embodiments, one channel may include the plurality of RF coil elements 220 and 230, and the plurality of RF coil elements 220 and 230 may be referred to as multiple loop-shaped RF coil elements. Since the RF surface coil unit 200 includes the multiple loop-shaped RF coil elements 220 and 230 that are electrically connected to each other and form one channel, mutual inductance coupling between the RF coil elements 220 and 230 may be ignored. In FIG. 2A, it is illustrated that the RF surface coil unit 200 includes four channels CH1, CH2, CH3, and CH4, but it is an example. When the multiple loop-shaped RF coil elements are included in one channel, a higher signal to noise ratio (SNR) may be obtained compared to when a single loop-shaped RF coil is included in one channel.

In addition, an additional RF coil element may further be formed in the loop structure of the second RF coil elements 230 in the RF surface coil unit 200. Also, an RF coil element having a larger loop structure than the first RF coil element 220 may further be formed at an outer side of the first RF coil element 220.

The base 210 of the RF surface coil unit 200 may include a non-magnetic material which is rigid and light and has excellent corrosion resistance and moldability. The base 210 may include, for example, an insulating polymer and a plastic material. FIG. 2A illustrates that the base 210 of the RF surface coil unit 200 has a quadrangular shape. However, the base 210 is not limited thereto, and may have a circular, oval, or other polygonal shape. Also, FIG. 2A illustrates that the base 210 of the RF surface coil unit 200 has a flat shape. However, it is an example, and the base 210 may be formed to have a shape of curved surface having a curvature. The RF coil elements 220 and 230 may include a conductive material. For example, the RF coil elements 220 and 230 may be formed by coating copper or a copper surface with a material having a high conductivity, such as silver or gold, but are not limited thereto. The RF coil elements 220 and 230 may be formed to have a thickness of about 3 mm to about 10 mm, but are not limited thereto.

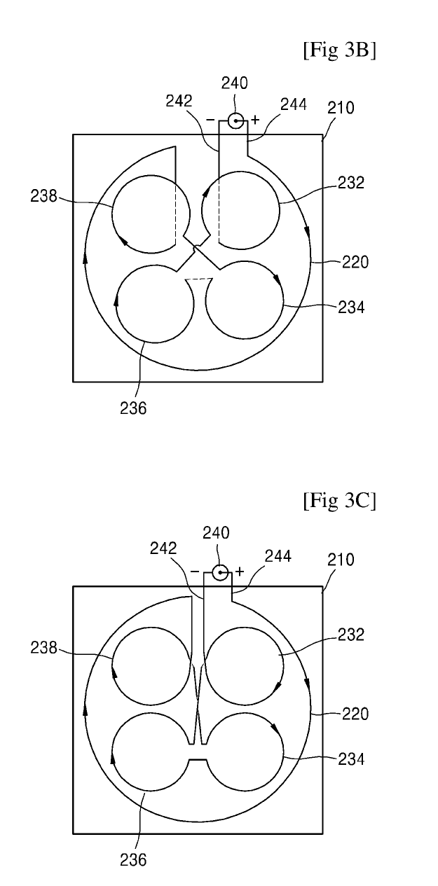

FIGS. 3A through 3C are views of a direction of currents of an RF surface coil unit of an MRI system, according to embodiments.

Referring to FIGS. 3A through 3C, one channel in the RF surface coil unit may include the first RF coil element 220 and second RF coil elements 232, 234, 236, and 238. The first RF coil element 220 and the second RF coil elements 232, 234, 236, and 238 may be connected to a coaxial cable 240. The RF surface coil unit may be connected to the signal transceiver 10 of FIG. 1 via the coaxial cable 240, to receive a control signal for forming a magnetic field or transmit a magnetic resonance signal obtained from an object to be examined. A positive terminal (+) of a portion in which the first RF coil element 220 and the second RF coil elements 232, 234, 236, and 238 are connected to the coaxial cable 240 may be connected to a signal line, and a negative terminal (−) of the portion may be connected to ground. Electrode lines 242 and 244 may be formed among the coaxial cable 240, the first RF coil element 220, and the second RF coil elements 232, 234, 236, and 238. FIG. 3A illustrates an example in which the first RF coil element 220 and the second RF coil elements 232, 234, 236, and 238 have opposite directions (arrows) of current flows. That is, the first RF coil element 220 may have a current flow of a clockwise direction, and the second RF coil elements 232, 234, 236, and 238 may have a current flow of a counter clockwise direction. FIGS. 3B and 3C illustrate examples in which the first RF coil element 220 and the second RF coil elements 232, 234, 236, and 238 have the same directions of current flows. That is, both of the first RF coil element 220 and the second RF coil elements 232, 234, 236, and 238 may show a current flow of a clockwise direction.

In the MRI system according to the embodiments, the multiple loop-shaped RF coil elements may form one channel. The RF coil according to the embodiments may include a plurality of channels, and each of the plurality of channels may include multiple loop-shaped coil elements.

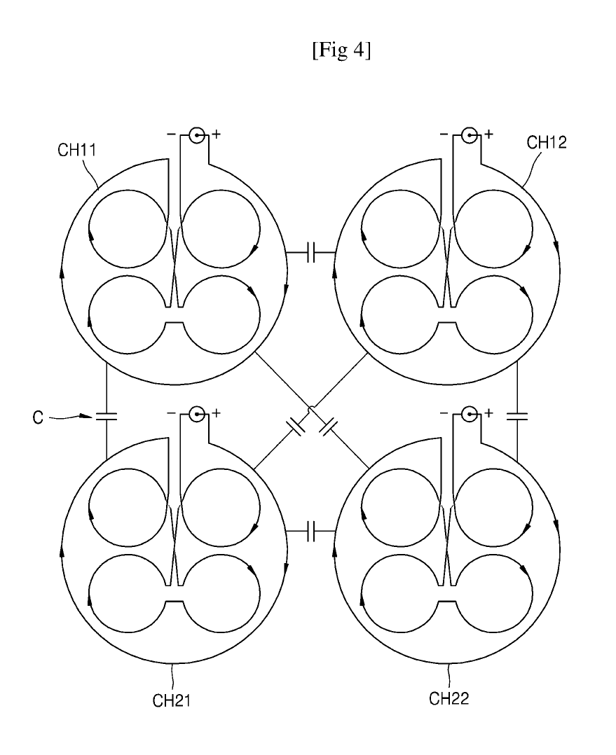

FIG. 4 is a view of an example in which an RF surface coil unit of an MRI system includes four channels CH11, CH12, CH13, and CH14, according to an embodiment. Referring to FIG. 4, the RF surface coil unit may include the four channels CH11, CH12, CH13, and CH14, and a decoupling device for decoupling between coil elements of other channels may be formed between each of the channels CH11, CH12, CH13, and CH14. For example, the decoupling device may be a capacitor C, or a decoupling circuit such as an inductor L or a transformer. The capacitor C, or the decoupling circuit such as the inductor L or the transformer may be used for decoupling.

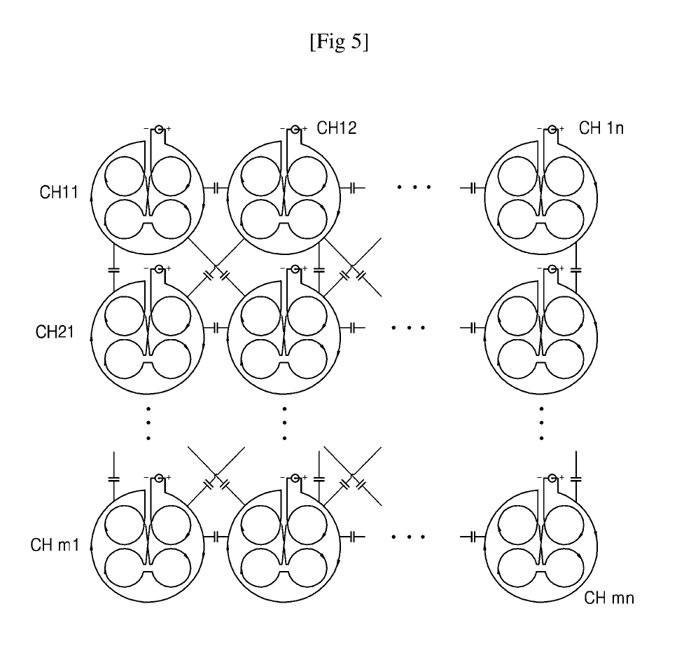

The RF surface coil unit of the MRI system according to an embodiment may be expanded to have four or more channels. For example, the RF surface coil unit of the MRI system may be expanded to have 8 through 128 channels. FIG. 5 illustrates an RF surface coil unit which is expanded to have multiple channels. Referring to FIG. 5, the RF surface coil unit may include the channels including m×n multiple loop-shaped RF coil elements.

The MRI system according to the embodiments has the multiple loop-shaped RF coil elements formed as one channel, and may include the RF surface coil unit which is expanded to have a plurality of channels. The MRI system according to the embodiments includes the RF coil elements having multiple loop structures in one channel, and thus, may improve sensitivity and uniformity compared to a case in which RF coil elements have a single loop structure. The RF surface coil unit according to the embodiments may be applied to a transmission and reception (Tx/Rx) or reception exclusive (Rx) MRI RF coil.

It should be understood that the embodiments described herein should be considered in a descriptive sense only and not for purposes of limitation. Descriptions of features or aspects within each embodiment should typically be considered as available for other similar features or aspects in other embodiments.

While one or more embodiments have been described with reference to the figures, it will be understood by those of ordinary skill in the art that various changes in form and details may be made therein without departing from the spirit and scope as defined by the following claims.

1. A radio frequency (RF) surface coil unit for a magnetic resonance imaging (MRI) system, the RF surface coil unit comprising multiple RF coil elements comprising:
at least one first RF coil element having a loop shape; and
at least one second RF coil element formed in the at least one first RF coil element.

2. The RF surface coil unit of claim 1, wherein the at least one first RF coil element and the at least one second RF coil element are electrically connected to each other, and the multiple RF coil elements comprising the at least one first RF coil element and the at least one second RF coil element are formed as one channel.

3. The RF surface coil unit of claim 2, comprising at least one channel comprising the multiple RF coil elements.

4. The RF surface coil unit of claim 3, wherein the at least one channel is formed as a plurality of channels, and a decoupling device is formed for decoupling between the coil elements of the at least one channel.

5. The RF surface coil unit of claim 4, wherein the decoupling device comprises a capacitor.

6. The RF surface coil unit of claim 5, wherein the decoupling device further comprises a decoupling circuit formed as an inductor or a transformer.

7. The RF surface coil unit of claim 4, wherein the decoupling device comprises a decoupling circuit formed as an inductor or a transformer.

8. The RF surface coil unit of claim 1, wherein the at least one first RF coil element has a circular shape, an oval shape, or a polygonal shape.

9. The RF surface coil unit of claim 1, wherein the at least one second RF coil element has a circular shape, an oval shape, or a polygonal loop shape.

10. A magnetic resonance imaging (MRI) system comprising:
a radio frequency (RF) surface coil unit comprising multiple RF coil elements comprising:
at least one first RF coil element having a loop shape; and
at least one second RF coil element formed in the at least one first RF coil element.

11. The MRI system of claim 10, wherein the at least one first RF coil element and the at least one second RF coil element are electrically connected to each other, and
the multiple RF coil elements comprising the at least one first RF coil element and the at least one second RF coil element are formed as one channel.

12. The MRI system of claim 11, comprising at least one channel comprising the multiple RF coil elements.

13. The MRI system of claim 12, wherein the at least one channel is formed as a plurality of channels, and a decoupling device is formed for decoupling between the coil elements of the at least one channel.
